(19) United States
(12) Patent Application Publication
(10) Pub. No.: US 2015/0118175 A1
(43) Pub. Date: Apr. 30, 2015

(54) COSMETIC COMPOSITION COMPRISING A FATTY-CHAIN SILANE AND A PARTICULAR FIXING POLYMER

(71) Applicant: L'OREAL, Paris (FR)

(72) Inventors: Laetitia Feuillette, Paris (FR); Gregory Plos, Paris (FR)

(21) Appl. No.: 14/396,981

(22) PCT Filed: Apr. 26, 2013
(86) PCT No.: PCT/EP2013/058713

§ 371 Date: Oct. 24, 2014

Related U.S. Application Data
(60) Provisional application No. 61/668,059, filed on Jul. 5, 2012.
(60) Provisional application No. 61/668,122, filed on Jul. 5, 2012.

(30) Foreign Application Priority Data
Apr. 26, 2012 (FR) 1253832
Apr. 26, 2012 (FR) 1253833

(51) Int. Cl. A61K 8/58; A61K 8/81; A61Q 5/06

(52) U.S. Cl. CPC A61K 8/585; A61Q 5/06; A61K 8/8135; A61K 8/8158; A61K 8/8152; A61K 8/8176; A61K 8/8182; A61K 8/8129
USPC 424/70.11

(57) ABSTRACT

The present invention relates to a cosmetic composition comprising: from 0.5% to 15% by weight, relative to the total weight of the composition of one or more fatty-chain silanes of formula (I) below and/or oligomers thereof: R₁Si(OR₂)_{z}(R₃)ₓ(OH)_{y} (I) in which formula (I): R₁ represents a linear or branched alkyl or alkenyl group comprising from 7 to 18 carbon atoms, R₂ and R₃, which may be identical or different, represent a linear or branched alkyl group comprising from 1 to 6 carbon atoms, y denotes an integer ranging from 0 to 3, z denotes an integer ranging from 0 to 3, preferentially from 1 to 3 and better still equal to 3, and x denotes an integer ranging from 0 to 2, with z+x+y=3, one or more fixing polymers chosen from anionic, amphoteric and non-ionic fixing polymers. The invention also relates to the use of said composition for shaping and/or fixing keratin fibres, such as human keratin fibres and in particular the hair, and also to a cosmetic treatment process for keratin fibres using such a composition.

The present invention relates to a cosmetic composition comprising one or more fatty-chain silanes and one or more particular fixing polymers.

A subject of the present invention is also the use of said composition for shaping and/or fixing keratin fibres, such as human keratin fibres and in particular the hair, and also a cosmetic treatment process for keratin fibres using such a composition.

Styling products are normally used to construct and structure the hairstyle and give it long-lasting form retention. They are usually in the form of lotions, gels, foams, creams, sprays, etc. The corresponding compositions generally comprise one or more fixing polymers, in a cosmetically acceptable medium. These polymers allow the formation of a coating film on the hair, thus providing form retention of the hairstyle.

However, the presence of these fixing polymers generally leads to the hair having a dry feel, before or after disentangling, which can create a perception of hair which has been damaged by these styling products.

There is therefore a need for compositions which enable strong fixing of the hair and a satisfactory cosmetic feel.

International patent application WO 2004/012 691 discloses the cosmetic use of silanes for improving the condition of the hair.

Patent application EP 0 159 628 proposes compositions for reinforcing the elasticity of the hair, comprising an alkyltrialkoxysilane.

Moreover, patent application EP 1 736 139 describes a hair treatment composition comprising an alkoxysilane, an organic acid and water, the pH of the composition being between 2 and 5.

Finally, patent application EP 0 877 027 discloses a composition comprising an organosilane and a particular polyol.

The Applicant has now discovered, surprisingly, that the combination of a particular fatty-chain silane with at least one anionic, amphoteric or non-ionic fixing polymer makes it possible to provide strong fixing of the hair and an improved cosmetic feel.

In particular, such a composition makes it possible, on the one hand, to confer good fixing on the head of hair and, on the other hand, to confer on the hair a natural soft feel, and a smooth feel after disentangling.

A subject of the present invention is thus a cosmetic composition comprising:
from 0.5% to 15% by weight, relative to the total weight of the composition of one or more fatty-chain silanes of formula (I) below and/or oligomers thereof:

R₁Si(OR₂)_{z}(R₃)ₓ(OH)_{y} (I)

in which formula (I):
R₁ represents a linear or branched alkyl or alkenyl group comprising from 7 to 18 carbon atoms,
R₂ and R₃, which may be identical or different, represent a linear or branched alkyl group comprising from 1 to 6 carbon atoms,
y denotes an integer ranging from 0 to 3, and
z denotes an integer ranging from 0 to 3, and
x denotes an integer ranging from 0 to 2,
with z+x+y=3,
one or more fixing polymers chosen from anionic, amphoteric and non-ionic fixing polymers.

Another subject of the invention consists of a cosmetic composition which can be obtained from one or more fixing polymers chosen from anionic, amphoteric and non-ionic fixing polymers, and from one or more compounds of formula (I) and/or oligomers thereof.

This particular combination makes it possible to obtain both strong fixing of the hair and form retention of the hairstyle, without introducing a dry feel or any greasy appearance or feel. The composition according to the invention also makes it possible to give the fibres a natural soft feel. It also makes it possible to confer a smooth, natural and soft feel on the fibres after disentangling.

Another subject of the invention consists of a cosmetic hair treatment process using a composition according to the invention.

Yet another subject of the invention is the use of the composition according to the invention for shaping and/or fixing the hairstyle.

Other subjects, characteristics, aspects and advantages of the invention will become even more clearly apparent on reading the description and examples that follow.

The fatty-chain silane(s) that may be used in the composition according to the invention are those corresponding to formula (I) below:

R₁Si(OR₂)_{z}(R₃)ₓ(OH)_{y} (I)

in which:
R₁ represents a linear or branched alkyl or alkenyl group comprising from 7 to 18 carbon atoms,
R₂ and R₃, which may be identical or different, represent a linear or branched alkyl group comprising from 1 to 6 carbon atoms,
y denotes an integer ranging from 0 to 3,
z denotes an integer ranging from 0 to 3, and
x denotes an integer ranging from 0 to 2,
with z+x+y=3.

The term “oligomer” means the polymerization products of the compounds of formula (I) comprising from 2 to 10 silicon atoms.

Preferably, R₃ represents an alkyl group comprising from 1 to 4 carbon atoms, better still a linear alkyl group comprising from 1 to 4 carbon atoms, and preferably the methyl or ethyl groups.

Preferably, R₂ represents an alkyl group comprising from 1 to 4 carbon atoms, better still a linear alkyl group comprising from 1 to 4 carbon atoms, and preferably the ethyl group.

Preferably, R₁ represents an alkyl group, more preferentially a linear alkyl group and even more preferentially an octyl group.

Preferably, z ranges from 1 to 3.

Even more preferentially, z is equal to 3.

Preferably, the composition comprises at least one fatty-chain silane chosen from octyltriethoxysilane, dodecyltriethoxysilane, octadecyltriethoxysilane and hexadecyltriethoxysilane.

More particularly, the composition comprises at least octyltriethoxysilane (OTES).

The silane(s) of formula (I) and/or oligomers thereof are present in the composition according to the invention in preferential proportions ranging from 1% to 15% by weight relative to the total weight of the composition.

The composition according to the invention also comprises one or more anionic, amphoteric or non-ionic fixing polymers.

For the purposes of the invention, the term “fixing polymer” means any polymer that is capable, by application to the hair, of giving a shape to the head of hair or of holding the hair in an already acquired shape.

The anionic fixing polymers generally used are polymers comprising groups derived from carboxylic acid, sulfonic acid or phosphoric acid and have a number-average molecular weight of between approximately 500 and 5 000 000.

The carboxylic groups are provided by unsaturated monocarboxylic or dicarboxylic acid monomers such as those corresponding to the formula:

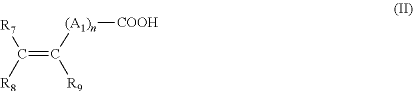

in which n is an integer from 0 to 10, A₁ denotes a methylene group, optionally connected to the carbon atom of the unsaturated group or to the neighbouring methylene group when n is greater than 1, via a heteroatom such as oxygen or sulfur, R₇ denotes a hydrogen atom, or a phenyl or benzyl group, R₈ denotes a hydrogen atom or a lower alkyl or carboxyl group, R₉ denotes a hydrogen atom, a lower alkyl group or a —CH₂—COOH, phenyl or benzyl group.

In the abovementioned formula, a lower alkyl group preferably denotes a group having 1 to 4 carbon atoms, and in particular the methyl and ethyl groups.

The anionic or amphoteric fixing polymers containing carboxylic groups that are preferred according to the invention are:

A) Copolymers of acrylic or methacrylic acid or salts thereof.

Among these polymers, mention may be made of copolymers of acrylic or methacrylic acid with a monoethylenic monomer such as ethylene, styrene, vinyl esters, acrylic or methacrylic acid esters, optionally grafted onto a polyalkylene glycol such as polyethylene glycol and optionally crosslinked. Such polymers are described in particular in French patent No. 1 222 944 and German application No. 2 330 956, the copolymers of this type comprising an optionally N-alkylated and/or hydroxyalkylated acrylamide unit in their chain as described in particular in Luxembourg patent application Nos. 75370 and 75371. Mention may also be made of copolymers of acrylic acid and of C₁-C₄ alkyl methacrylate and terpolymers of vinylpyrrolidone, of acrylic acid and of C₁-C₂₀ alkyl methacrylate, for example lauryl methacrylate, such as the product sold by the company ISP under the name Acrylidone® LM (INCI name VP/acrylates/lauryl methacrylate copolymer), acrylic acid/ethyl acrylate/N-t-butylacrylamide terpolymers, such as the products Ultrahold® Strong and Ultrahold® 8 sold by the company BASF (INCI name Acrylates/t-butylacrylamide copolymer), methacrylic acid/ethyl acrylate/tert-butyl acrylate terpolymers, such as the products sold under the names Luvimer® 100 P or Luvimer® PRO 55 by the company BASF (INCI name Acrylates copolymer), copolymers of methacrylic acid and of ethyl acrylate, such as the products sold under the names Luvimer® MAE or Luviflex® Soft by the company BASF (INCI name Acrylates copolymer), acrylic acid/butyl acrylate/methyl methacrylate terpolymers, such as the product sold under the name Balance® CR by the company Akzo Nobel (INCI name Acrylates copolymer), and the copolymers of methacrylic acid and of methyl methacrylate sold under the name Eudragit® L 100 by the company Rohm Pharma (INCI name Acrylates copolymer). Mention may also be made of branched block polymers containing (meth)acrylic acid monomers, such as the product sold under the name Fixate® G-100L by the company Lubrizol (INCI name AMP-acrylates/allyl methacrylate copolymer). Mention may also be made of acrylate/acrylate hydroxy ester copolymers, such as Acudyne 180 sold by the company Dow Chemical.

B) Crotonic acid copolymers, such as those comprising vinyl acetate or propionate units in their chain and optionally other monomers such as allyl esters or methallyl esters, vinyl ether or vinyl ester of a linear or branched saturated carboxylic acid with a long hydrocarbon-based chain, such as those containing at least 5 carbon atoms, it being possible for these polymers optionally to be grafted or crosslinked, or alternatively another vinyl, allyl or methallyl ester monomer of an α- or β-cyclic carboxylic acid. Such polymers are described, inter alia, in French patents Nos. 1 222 944, 1 580 545, 2 265 782, 2 265 781, 1 564 110 and 2 439 798. Commercial products which fall into this category are the products Resyn® 28-2930 and 28-1310 sold by the company Akzo Nobel (INCI names VA/crotonates/vinyl decanoate copolymer and VA/crotonates copolymer, respectively. Mention may also be made of the products Luviset® CA 66 sold by the company BASF, Aristoflex® A60 sold by the company Clariant (INCI name VA/crotonates copolymer) and Mexomere® PW or PAM sold by the company Chimex (INCI name VA/vinyl butyl benzoate/crotonates copolymer).

C) Copolymers of C₄-C₈ monounsaturated carboxylic acids or anhydrides chosen from:
copolymers comprising (i) one or more maleic, fumaric or itaconic acids or anhydrides and (ii) at least one monomer chosen from vinyl esters, vinyl ethers, vinyl halides, phenylvinyl derivatives, acrylic acid and its esters, the anhydride functions of these copolymers optionally being monoesterified or monoamidated. Such polymers are described, in particular, in U.S. Pat. Nos. 2,047,398, 2,723,248 and 2,102,113, and GB patent No. 839 805. Commercial products are in particular those sold under the names Gantrez® AN or ES by the company ISP, such as Gantrez® ES 225 (INCI name Ethyl ester of PVM/MA copolymer) or Gantrez® ES 425L (INCI name Butyl ester of PVM/MA copolymer);
copolymers comprising (i) one or more maleic, citraconic or itaconic anhydride units and (ii) one or more monomers chosen from allyl or methallyl esters optionally comprising one or more acrylamide, methacrylamide, α-olefin, acrylic or methacrylic ester, acrylic or methacrylic acid or vinylpyrrolidone groups in their chain,

the anhydride functions of these copolymers optionally being monoesterified or monoamidated.

These polymers are described, for example, in French patents Nos. 2 350 384 and 2 357 241 by the Applicant.

D) Polyacrylamides comprising carboxylate groups.

E) Polyurethanes optionally comprising silicones, with carboxylate groups, such as Luviset Pur and Luviset Si Pur provided by the company BASF.

The fixing polymers comprising units derived from sulfonic acid can be chosen from:

A′) homopolymers and copolymers comprising vinylsulfonic, styrenesulfonic, naphthalenesulfonic and acrylamidoalkylsulfonic units.

These polymers can be chosen in particular from:
polyvinylsulfonic acid salts having a molecular weight of approximately between 1000 and 100 000, and also the copolymers with an unsaturated comonomer such as acrylic or methacrylic acids and their esters, and also acrylamide or its derivatives, vinyl ethers and vinylpyrrolidone;
polystyrenesulfonic acid salts such as the sodium salts that are sold for example under the name Flexan® II by Akzo Nobel (INCI name Sodium polystyrene sulfonate). These compounds are described in patent FR 2 198 719;
polyacrylamidosulfonic acid salts, such as those mentioned in U.S. Pat. No. 4,128,631, and more particularly the polyacrylamidoethylpropanesulfonic acid, sold under the name Rheocare® HSP-1180 by Cognis (INCI name polyacrylamidomethylpropane sulfonic acid);

B′) sulfonic polyesters, these polymers being advantageously obtained by polycondensation of at least one dicarboxylic acid, of at least one diol or of a mixture of diol and of diamine, and of at least one difunctional monomer comprising a sulfonic function. Among these polymers, mention may be made of:
linear sulfonic polyesters such as those described in patent applications U.S. Pat. No. 3,734,874, U.S. Pat. No. 3,779,993, U.S. Pat. No. 4,119,680, U.S. Pat. No. 4,300,580, U.S. Pat. No. 4,973,656, U.S. Pat. No. 5,660,816, U.S. Pat. No. 5,662,893 and U.S. Pat. No. 5,674,479. Such polymers are, for example, the products Eastman® AQ38S Polymer, Eastman® AQ55S Polymer and Eastman® AQ48 Ultra Polymer sold by the company Eastman Chemical (name Polyester-5) which are copolymers obtained from diethylene glycol, from 1,4-cyclohexanedimethanol, from isophthalic acid and from sulfoisophthalic acid salt;
branched sulfonic polyesters such as those described in patent applications WO 95/18191, WO 97/08261 and WO 97/20899. Such compounds are, for example, the products Eastman® AQ10D Polymer (name Polyester-13)

or Eastman® AQ1350 Polymer provided by the company Eastman Chemical (name Polyester-13).

According to the invention, the anionic fixing polymers are preferably chosen from copolymers of acrylic acid, such as the acrylic acid/ethyl acrylate/N-tert-butylacrylamide terpolymers sold in particular under the name Ultrahold® Strong by the company BASF, copolymers derived from crotonic acid, such as vinyl acetate/vinyl tert-butylbenzoate/crotonic acid terpolymers and the crotonic acid/vinyl acetate/vinyl neododecanoate terpolymers sold in particular under the name Resyn 28-2930 by the company Akzo Nobel, polymers derived from maleic, fumaric or itaconic acids or anhydrides with vinyl esters, vinyl ethers, vinyl halides, phenylvinyl derivatives and acrylic acid and esters thereof, such as the methyl vinyl ether/monoesterified maleic anhydride copolymers sold, for example, under the names Gantrez® ES 425L or ES 225 by the company ISP, the copolymers of methacrylic acid and of ethyl acrylate sold under the name Luvimer® MAE by the company BASF, and the vinyl acetate/crotonic acid copolymers sold under the name Luviset® CA 66 by the company BASF, and the vinyl acetate/crotonic acid copolymers grafted with polyethylene glycol sold under the name Aristoflex® A60 by the company Clariant, the vinylpyrrolidone/acrylic acid/lauryl methacrylate terpolymers sold under the name Acrylidone® LM by the company ISP, the polymer sold under the name Fixate® G-100L by the company Lubrizol, the vinyl acetate/crotonic acid/vinyl p-tert-butylbenzoate copolymers sold under the names Mexomere® PW or PAM by the company Chimex, and acrylate/acrylate hydroxyester copolymers, such as Acudyne 180 sold by the company Dow Chemical.

The amphoteric fixing polymers that may be used in accordance with the invention may be chosen from polymers comprising units B and C distributed randomly in the polymer chain, where B denotes a unit derived from a monomer comprising at least one basic nitrogen atom and C denotes a unit derived from an acid monomer comprising one or more carboxylic or sulfonic groups, or alternatively B and C may denote groups derived from carboxybetaine or sulfobetaine zwitterionic monomers;

B and C can also denote a cationic polymer chain comprising primary, secondary, tertiary or quaternary amine groups, in which at least one of the amine groups bears a carboxylic or sulfonic group connected via a hydrocarbon-based group, or alternatively B and C form part of a chain of a polymer comprising an α,β-dicarboxylic ethylene unit in which one of the carboxylic groups has been made to react with a polyamine comprising one or more primary or secondary amine groups.

The amphoteric fixing polymers corresponding to the definition given above that are more particularly preferred are chosen from the following polymers:

(1) copolymers comprising acidic vinyl units and basic vinyl units, such as those resulting from the copolymerization of a monomer derived from a vinyl compound bearing a carboxylic group such as, more particularly, acrylic acid, methacrylic acid, maleic acid, α-chloroacrylic acid, and of a basic monomer derived from a substituted vinyl compound containing at least one basic atom, such as, more particularly, dialkylaminoalkyl methacrylate and acrylate, dialkylaminoalkylmethacrylamide and -acrylamide. Such compounds are described in U.S. Pat. No. 3,836,537;

(2) polymers comprising units deriving from:
a) at least one monomer chosen from acrylamides and methacrylamides substituted on the nitrogen atom with an alkyl group,
b) at least one acidic comonomer containing one or more reactive carboxylic groups, and
c) at least one basic comonomer such as esters containing primary, secondary, tertiary and quaternary amine substituents of acrylic and methacrylic acids and the product of quaternization of dimethylaminoethyl methacrylate with dimethyl or diethyl sulfate.

The N-substituted acrylamides or methacrylamides that are more particularly preferred according to the invention are compounds in which the alkyl groups contain from 2 to 12 carbon atoms and more particularly N-ethylacrylamide, N-tert-butylacrylamide, N-tert-octylacrylamide, N-octylacrylamide, N-decylacrylamide, N-dodecylacrylamide and the corresponding methacrylamides.

The acidic comonomers are chosen more particularly from acrylic, methacrylic, crotonic, itaconic, maleic and fumaric acid and alkyl monoesters, having 1 to 4 carbon atoms, of maleic or fumaric acid or anhydride.

The preferred basic comonomers are aminoethyl, butylaminoethyl, N,N′-dimethylaminoethyl and N-tert-butylaminoethyl methacrylates.

The copolymers whose INCI name is Octylacrylamide/acrylates/butylaminoethyl methacrylate copolymer, such as the products sold under the names Amphomer®, Amphomer® LV71 or Balance® 47 by the company Akzo Nobel, are particularly used;

(3) crosslinked and acylated polyaminoamides partially or totally deriving from polyaminoamides of general formula:

CO—R₁₀—CO—Z (III)

in which R₁₀ represents a divalent group derived from a saturated dicarboxylic acid, an aliphatic monocarboxylic or dicarboxylic acid containing an ethylenic double bond, an ester of a lower alkanol, having 1 to 6 carbon atoms, of these acids, or a group derived from the addition of any one of said acids to a bis(primary) or bis(secondary) amine, and Z denotes a group derived from a bis(primary), mono(secondary) or bis(secondary) polyalkylene-polyamine and preferably represents:
a) in proportions of from 60 to 100 mol %, the group:

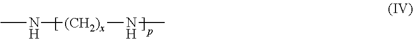

where x=2 and p=2 or 3, or alternatively x=3 and p=2,

this group deriving from diethylenetriamine, from triethylenetetraamine or from dipropylenetriamine;
b) in proportions of from 0 to 40 mol %, the group (IV) above in which x=2 and p=1 and which is derived from ethylenediamine, or the group deriving from piperazine:

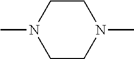

c) in proportions of from 0 to 20 mol %, the —NH—(CH₂)₆—NH— group deriving from hexamethylenediamine,

these polyaminoamides being crosslinked by addition reaction of a difunctional crosslinking agent chosen from epihalohydrins, diepoxides, dianhydrides and bis-unsaturated derivatives, using from 0.025 to 0.35 mol of crosslinking agent per amine group of the polyaminoamide and acylated by the action of acrylic acid, chloroacetic acid or an alkane sultone, or salts thereof.

The saturated carboxylic acids are preferably chosen from acids having 6 to 10 carbon atoms, such as adipic acid, 2,2,4-trimethyladipic acid and 2,4,4-trimethyladipic acid, terephthalic acid, acids containing an ethylenic double bond such as, for example, acrylic acid, methacrylic acid and itaconic acid.

The alkane sultones used in the acylation are preferably propane sultone or butane sultone; the salts of the acylating agents are preferably the sodium or potassium salts;

(4) polymers comprising zwitterionic units of formula:

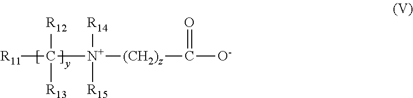

in which R₁₁ denotes a polymerizable unsaturated group such as an acrylate, methacrylate, acrylamide or methacrylamide group, y and z represent an integer from 1 to 3, R₁₂ and R₁₃ represent a hydrogen atom, a methyl, ethyl or propyl group, R₁₄ and R₁₅ represent a hydrogen atom or an alkyl group such that the sum of the carbon atoms in R₁₄ and R₁₅ does not exceed 10.

The polymers comprising such units can also comprise units derived from non-zwitterionic monomers such as dimethyl- or diethylaminoethyl acrylate or methacrylate or alkyl acrylates or methacrylates, acrylamides or methacrylamides, or vinyl acetate.

Mention may be made, by way of example, of methyl methacrylate/methyl dimethylcarboxymethylammonioethyl methacrylate copolymers, such as the product sold under the name Diaformer Z-301N or Z-301W by the company Clariant (INCI name Acrylates copolymer);

(5) polymers derived from chitosan comprising monomer units corresponding to the following formulae:

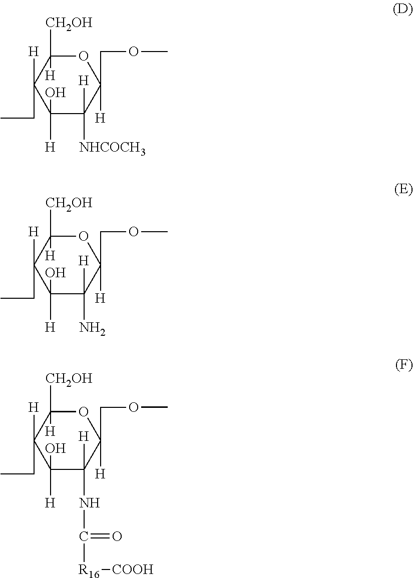

the unit (D) being present in proportions of between 0 and 30%, the unit (E) in proportions of between 5% and 50% and the unit (F) in proportions of between 30% and 90%, it being understood that, in this unit (F), R₁₆ represents a group of formula:

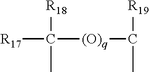

in which, if q=0, R₁₇, R₁₈ and R₁₉, which may be identical or different, each represent a hydrogen atom, a methyl, hydroxyl, acetoxy or amino residue, a monoalkylamine residue or a dialkylamine residue that are optionally interrupted by one or more nitrogen atoms and/or optionally substituted with one or more amine, hydroxyl, carboxyl, alkylthio or sulfonic groups, an alkylthio residue in which the alkyl group bears an amino residue, at least one of the groups R₁₇, R₁₈ and R₁₉ being, in this case, a hydrogen atom;

or, if q=1, R₁₇, R₁₈ and R₁₉ each represent a hydrogen atom, as well as the salts formed by these compounds with bases or acids;

(6) polymers corresponding to the general formula (VI) are described, for example, in French patent 1 400 366:

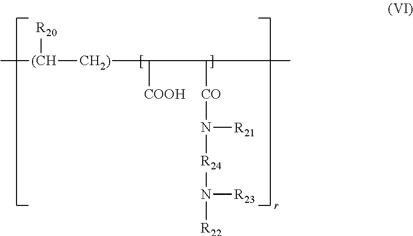

in which R₂₀ represents a hydrogen atom, a CH₃O, CH₃CH₂O or phenyl group, R₂₁ denotes a hydrogen atom or a lower alkyl group such as methyl or ethyl, R₂₂ denotes a hydrogen atom or a C₁₋₆ lower alkyl group such as methyl or ethyl, R₂₃ denotes a C₁₋₆ lower alkyl group such as methyl or ethyl or a group corresponding to the formula: —R₂₄—N(R₂₂)₂, R₂₄ representing a —CH₂—CH₂—, —CH₂—CH₂—CH₂— or —CH₂—CH(CH₃)— group, R₂₂ having the meanings mentioned above;

(7) polymers derived from the N-carboxyalkylation of chitosan, such as N-carboxymethyl chitosan or N-carboxybutyl chitosan, for instance the product sold under the name Chitoglycan by the company Sinerga SPA (INCI name Carboxymethyl chitosan);

(8) amphoteric polymers of the -D-X-D-X type chosen from:
a) polymers obtained by the action of chloroacetic acid or sodium chloroacetate on compounds comprising at least one unit of formula:

-D-X-D-X-D- (VII)

where D denotes a group

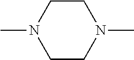

and X denotes the symbol E or E′, E or E′, which may be identical or different, denote a divalent group that is an alkylene group with a straight or branched chain containing up to 7 carbon atoms in the main chain, which is unsubstituted or substituted with hydroxyl groups and which can comprise, in addition to the oxygen, nitrogen and sulfur atoms, 1 to 3 aromatic and/or heterocyclic rings; the oxygen, nitrogen and sulfur atoms being present in the form of ether, thioether, sulfoxide, sulfone, sulfonium, alkylamine or alkenylamine groups, hydroxyl, benzylamine, amine oxide, quaternary ammonium, amide, imide, alcohol, ester and/or urethane groups;
b) polymers of formula:

-D-X-D-X— (VII′)

where D denotes a

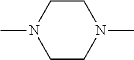

and X denotes the symbol E or E′ and at least once E′; E having the meaning given above and E′ is a divalent group that is an alkylene group with a straight or branched chain having up to 7 carbon atoms in the main chain, which is unsubstituted or substituted with one or more hydroxyl groups and containing one or more nitrogen atoms, the nitrogen atom being substituted with an alkyl chain that is optionally interrupted by an oxygen atom and necessarily comprising one or more carboxyl functions or one or more hydroxyl functions and betainized by reaction with chloroacetic acid or sodium chloroacetate;

(9) (C₁-C₅)alkyl vinyl ether/maleic anhydride copolymers partially modified by semiamidation with an N,N-dialkylaminoalkylamine such as N,N-dimethylaminopropylamine or by semiesterification with an N,N-dialkylaminoalkanol. These copolymers can also comprise other vinyl comonomers such as vinylcaprolactam.

Among the amphoteric fixing polymers mentioned above that are most particularly preferred according to the invention, mention will be made of those of family (3), such as the copolymers of which the INCI name is Octylacrylamide/acrylates/butylaminoethyl methacrylate copolymer, such as the products sold under the names Amphomer®, Amphomer® LV 71 or Balance® 47 by the company Akzo Nobel and those of family (4), such as the methyl methacrylate/methyl dimethylcarboxymethylammonioethyl methacrylate copolymers sold, for example, under the name Diaformer Z-301N or Z-301W by the company Clariant.

The non-ionic fixing polymers result from a polymerization synthesis reaction. They are different from natural fixing polymers and from modified natural fixing polymers.

The non-ionic fixing polymers that may be used according to the present invention are chosen, for example, from:
polyalkyloxazolines;
vinyl acetate homopolymers;
vinyl acetate copolymers, for instance copolymers of vinyl acetate and of acrylic ester, copolymers of vinyl acetate and of ethylene, copolymers of vinyl acetate and of maleic ester, for example of dibutyl maleate, vinyl acetate/vinyl alcohol copolymers in particular resulting from the partial hydrolysis of vinyl acetate;
homopolymers and copolymers of acrylic esters, for instance copolymers of alkyl acrylates and of alkyl methacrylates, such as the products provided by the company Rohm GmbH under the name Eudragit® NE 30 D (INCI name Acrylates copolymer);
copolymers of acrylonitrile and of a non-ionic monomer, chosen, for example, from butadiene and alkyl (meth)acrylates;
styrene homopolymers;
styrene copolymers, for instance copolymers of styrene, of alkyl acrylate and of alkyl methacrylate; copolymers of styrene and of butadiene, or copolymers of styrene, of butadiene and of vinylpyridine;
polyamides;
vinyllactam homopolymers, such as the vinylpyrrolidone homopolymers sold, for example, under the names Luviskol® K30 powder by the company BASF or PVP K30L or K60 solution or K90 by the company ISP, or such as the polyvinylcaprolactam sold under the name Luviskol® Plus by the company BASF (INCI name PVP);
vinyllactam copolymers, such as a poly(vinylpyrrolidone/vinyllactam) copolymer sold under the trade name Luvitec® VPC 55K65W by the company BASF, poly(vinylpyrrolidone/vinyl acetate) copolymers, such as those sold under the name PVP/VA® S630L, E735, E635 and W735 by the company ISP, Luviskol® VA 73, VA 64 and VA 37 by the company BASF (INCI name VP/VA copolymer); and vinylpyrrolidone/methacrylamide/vinylimidazole terpolymers, for instance the product sold under the name Luviset® Clear by the company BASF (INCI name VP/methacrylamide/vinyl imidazole copolymer);
non-hydrolyzed polyvinylformamide polymers,
homopolymers of vinyl alcohol or polyvinyl alcohols resulting from the total hydrolysis of vinyl acetate, such as the products sold by the company Celanese under the names Celvol 205, 523, 540, 805, 823 and 840.

The alkyl groups of the abovementioned non-ionic polymers preferably have from 1 to 6 carbon atoms.

The anionic, amphoteric or non-ionic fixing polymer(s) are present in the composition according to the invention in preferential proportions ranging from 0.1% to 50% by weight, more preferentially from 0.5% to 20% by weight and better still from 1% to 15% by weight, relative to the total weight of the composition.

In one preferred embodiment, the weight ratio between, on the one hand, the amount of fatty-chain silane(s) of formula (I) and/or oligomers thereof, and on the other hand, the amount of anionic, amphoteric or non-ionic fixing polymer(s) ranges from 0.05 to 20, more preferably from 0.1 to 10 and better still from 0.2 to 5.

According to one embodiment, the weight ratio between, on the one hand, the amount of fatty-chain silane(s) of formula (I) and/or oligomers thereof, and on the other hand, the amount of anionic, amphoteric or non-ionic fixing polymer(s) is greater than or equal to 1.

The composition according to the invention may also comprise at least one surfactant.

The surfactants are chosen from anionic, cationic, non-ionic, amphoteric and zwitterionic surfactants.

The term “anionic surfactant” means a surfactant comprising, as ionic or ionizable groups, only anionic groups. These anionic groups are preferably chosen from the groups —C(O)OH, —C(O)O⁻, —SO₃H, —S(O)₂O⁻, —OS(O)₂OH, —OS(O)₂O⁻, —P(O)OH₂, —P(O)₂O⁻, —P(O)O₂⁻, —P(OH)₂, ═P(O)OH, —P(OH)O⁻, ═P(O)O⁻, ═POH and ═PO⁻, the anionic parts comprising a cationic counterion such as an alkali metal, an alkaline-earth metal or an ammonium.

By way of examples of anionic surfactants that may be used in the composition according to the invention, mention may be made of alkyl sulfates, alkyl ether sulfates, alkylamido ether sulfates, alkylarylpolyether sulfates, monoglyceride sulfates, alkylsulfonates, alkylamidesulfonates, alkylarylsulfonates, α-olefin sulfonates, paraffin sulfonates, alkylsulfosuccinates, alkylether sulfosuccinates, alkylamide sulfosuccinates, alkylsulfoacetates, acylsarcosinates, acylglutamates, alkylsulfosuccinamates, acylisethionates and N-acyltaurates, salts of alkyl monoesters of polyglycoside-polycarboxylic acids, acyllactylates, D-galactoside uronic acid salts, alkyl ether carboxylic acid salts, alkylaryl ether carboxylic acid salts, alkylamido ether carboxylic acid salts; and the corresponding non-salified forms of all these compounds; the alkyl and acyl groups of all these compounds comprising from 6 to 40 carbon atoms and the aryl group denoting a phenyl group.

These compounds can be oxyethylenated and then preferably comprise from 1 to 50 ethylene oxide units.

The salts of C₆-C₂₄ alkyl monoesters of polyglycoside-polycarboxylic acids can be chosen from C₆-C₂₄ alkyl polyglycoside-citrates, C₆-C₂₄ alkyl polyglycoside-tartrates and C₆-C₂₄ alkyl polyglycoside-sulfosuccinates.

When the anionic surfactant(s) are in salt form, it (they) may be chosen from alkali metal salts such as the sodium or potassium salt and preferably the sodium salt, ammonium salts, amine salts and in particular amino alcohol salts or alkaline-earth metal salts such as the magnesium salts.

By way of examples of amino alcohol salts, mention may in particular be made of monoethanolamine, diethanolamine and triethanolamine salts, monoisopropanolamine, diisopropanolamine or triisopropanolamine salts, 2-amino-2-methyl-1-propanol salts, 2-amino-2-methyl-1,3-propanediol salts and tris(hydroxymethyl)aminomethane salts.

Alkali metal or alkaline-earth metal salts, and in particular sodium or magnesium salts, are preferably used.

Use is preferably made, among the anionic surfactants mentioned, of (C₆-C₂₄)alkyl sulfates, (C₆-C₂₄)alkyl ether sulfates comprising from 2 to 50 ethylene oxide units, in particular in the form of alkali metal, ammonium, amino alcohol and alkaline-earth metal salts, or a mixture of these compounds.

In particular, it is preferable to use (C₁₂-C₂₀)alkyl sulfates, (C₁₂-C₂₀)alkyl ether sulfates comprising from 2 to 20 ethylene oxide units, in particular in the form of alkali metal, ammonium, amino alcohol and alkaline-earth metal salts, or a mixture of these compounds. Better still, it is preferred to use sodium lauryl ether sulfate containing 2.2 mol of ethylene oxide.

Examples of additional non-ionic surfactants that may be used in the compositions of the present invention are described, for example, in the “Handbook of Surfactants” by M. R. Porter, published by Blackie & Son (Glasgow and London), 1991, pp 116-178. They are in particular chosen from alcohols, α-diols and (C₁-C₂₀)alkylphenols, these compounds being etherified with ethoxylated, propoxylated or glycerolated groups and containing at least one fatty chain comprising, for example, from 8 to 40 carbon atoms, the number of ethylene oxide and/or propylene oxide groups possibly ranging in particular from 1 to 200, and the number of glycerol groups possibly ranging in particular from 1 to 30.

Mention may also be made of condensates of ethylene oxide and of propylene oxide with fatty alcohols; polyethoxylated fatty amides preferably having from 2 to 30 ethylene oxide units, polyglycerolated fatty amides containing on average from 1 to 5 and in particular from 1.5 to 4 glyerol groups, ethoxylated fatty acid esters of sorbitan containing from 2 to 30 ethylene oxide units, fatty acid esters of sucrose, fatty acid esters of polyethylene glycol, (C₆-C₂₄ alkyl)polyglycosides, N—(C₆-C₂₄ alkyl)glucamine derivatives, amine oxides such as (C₁₀C₁₄ alkyl)amine oxides or N—(C₁₀-C₁₄ acyl)aminopropylmorpholine oxides.

The amphoteric or zwitterionic surfactants which can be used in the present invention may in particular be secondary or tertiary aliphatic amine derivatives in which the aliphatic group is a linear or branched chain containing from 8 to 22 carbon atoms, said amine derivatives containing at least one anionic group, for instance a carboxylate, sulfonate, sulfate, phosphate or phosphonate group. Mention may be made in particular of (C₈-C₂₀ alkyl)betaines, sulfobetaines, (C₈-C₂₀ alkyl)amido(C₆₋₈ alkyl)betaines and (C₈-C₂₀ alkyl)amido(C₆-C₈ alkyl)sulfobetaines.

Among the optionally quaternized secondary or tertiary aliphatic amine derivatives that can be used, as defined above, mention may also be made of:

the compounds classified in the CTFA dictionary, 5th edition, 1993, under the names disodium cocoamphodiacetate, disodium lauroamphodiacetate, disodium caprylamphodiacetate, disodium capryloamphodiacetate, disodium cocoamphodipropionate, disodium lauroamphodipropionate, disodium caprylamphodipropionate, disodium capryloamphodipropionate, lauroamphodipropionic acid and cocoamphodipropionic acid.

By way of example, mention may be made of the cocoamphodiacetate sold by the company Rhodia under the trade name Miranol® C2M Concentrate.

Use may also be made of the compound classified in the CTFA dictionary under the name sodium diethylaminopropyl cocoaspartamide and sold by the company Chimex under the name Chimexane HB.

Among the amphoteric or zwitterionic surfactants mentioned above, use is preferably made of (C₈-C₂₀)alkylbetaines such as cocobetaine, (C₈-C₂₀)alkylamido(C₃-C₈)alkylbetaines such as cocamidopropylbetaine, the compounds of formula (B′2), such as the sodium salt of diethylaminopropyl laurylaminosuccinamate (INCI name: sodium diethylaminopropyl cocoaspartamide) and mixtures thereof. More preferentially, the amphoteric or zwitterionic surfactant(s) are chosen from cocamidopropylbetaine and cocobetaine.

The cationic surfactant(s) which can be used in the composition according to the invention comprise, for example, salts of optionally polyoxyalkylenated primary, secondary or tertiary fatty amines, quaternary ammonium salts, and mixtures thereof.

Examples of quaternary ammonium salts that may in particular be mentioned include:
tetraalkylammonium chlorides, such as, for example, dialkyldimethylammonium or alkyltrimethylammonium chlorides in which the alkyl group comprises approximately from 12 to 22 carbon atoms, in particular behenyltrimethylammonium, distearyldimethylammonium, cetyltrimethylammonium or benzyldimethylstearylammonium chlorides, or else, secondly, distearoylethylhydroxyethylmethylammonium methosulfate, dipalmitoylethylhydroxyethylammonium methosulfate or distearoylethylhydroxyethylammonium methosulfate, or else, lastly, palmitylamidopropyltrimethylammonium chloride or stearamidopropyldimethyl(myristyl acetate)ammonium chloride, sold under the name Ceraphyl® 70 by the company Van Dyk;
quaternary ammonium salts of imidazoline, for instance the product sold under the name Rewoquat® W 75 by the company Rewo;
diquaternary or triquaternary ammonium salts, for instance Finquat CT-P, provided by the company Finetex (Quaternium 89), and Finquat CT, provided by the company Finetex (Quaternium 75);
quaternary ammonium salts containing one or more ester functions, such as the salts, in particular the chloride or methyl sulfate, of diacyloxyethyldimethylammonium, diacyloxyethylhydroxyethylmethylammonium, monoacyloxyethyldihydroxyethylmethylammonium, triacyloxyethylmethylammonium or monoacyloxyethylhydroxyethyldimethylammonium, and mixtures thereof. The acyl groups preferably contain 14 to 18 carbon atoms and are obtained more particularly from a plant oil, such as palm oil or sunflower oil. When the compound comprises several acyl groups, these groups may be identical or different.

These products are obtained, for example, by direct esterification of triethanolamine, triisopropanolamine, an alkyldiethanolamine or an alkyldiisopropanolamine, which are optionally oxyalkylenated, with fatty acids or with fatty acid mixtures of plant or animal origin, or by transesterification of the methyl esters thereof. This esterification is followed by a quaternization by means of an alkylating agent such as an alkyl halide, preferably methyl or ethyl halide, a dialkyl sulfate, preferably dimethyl or diethyl sulfate, methyl methanesulfonate, methyl para-toluenesulfonate, glycol chlorohydrin or glycerol chlorohydrin.

Such compounds are sold, for example, under the names Dehyquart® by the company Henkel, Stepanquat® by the company Stepan, Noxamium® by the company Ceca or Rewoquat® WE 18 by the company Rewo-Witco.

The composition according to the invention may contain, for example, a mixture of quaternary ammonium salts of monoesters, diesters and triesters with a weight majority of diester salts.

Use may also be made of the ammonium salts containing at least one ester function that are described in patents U.S. Pat. No. 4,874,554 and U.S. Pat. No. 4,137,180.

Use may be made of behenoylhydroxypropyltrimethylammonium chloride, sold by Kao under the name Quatarmin BTC 131.

Preferably, the ammonium salts containing at least one ester function contain two ester functions.

Among the cationic surfactants that may be present in the composition according to the invention, it is more particularly preferred to choose cetyltrimethylammonium, behenyltrimethylammonium and dipalmitoylethylhydroxyethylmethylammonium salts, and mixtures thereof, and more particularly behenyltrimethylammonium chloride, cetyltrimethylammonium chloride and dipalmitoylethylhydroxyethylammonium methosulfate, and mixtures thereof.

When they are present, the surfactants may represent from 0.01% to 30%, preferably from 0.1% to 20% by weight and better still from 1% to 10% by weight, relative to the total weight of the composition.

In addition, the composition according to the invention may contain at least one fatty substance.

For the purposes of the present invention, the term “fatty substance” is intended to mean an organic compound which is insoluble in water at normal temperature (25° C.) and at atmospheric pressure (760 mmHg, i.e. 1.013×10⁵ Pa), that is to say with a solubility of less than 4% by weight, preferably of less than 1% by weight and even more preferentially of less than 0.1% by weight. They exhibit, in their structure, at least one hydrocarbon-based chain comprising at least 6 carbon atoms or a sequence of at least two siloxane groups. In addition, the fatty substances are soluble in organic solvents under the same temperature and pressure conditions, for instance chloroform, ethanol or benzene.

The fatty substances are not etherified with oxyalkylenated or glycerolated groups.

The fatty substances of the invention may be liquid or non-liquid at ambient temperature (25° C.) and at atmospheric pressure (760 mmHg; i.e. 1.013×10⁵ Pa).

The liquid fatty substances of the invention preferably have a viscosity of less than or equal to 2 Pa·s, better still less than or equal to 1 Pa·s and even better still less than or equal to 0.1 Pa·s at a temperature of 25° C. and at a shear rate of 1 s⁻¹.

Liquid fatty substances that may be mentioned include silicone or non-silicone oils.

The term “non-silicone oil” means an oil not containing any silicon atoms (Si) and the term “silicone oil” means an oil containing at least one silicon atom.

More particularly, the fatty substances are chosen from C₆-C₁₆ hydrocarbons, hydrocarbons containing more than 16 carbon atoms, non-silicone oils of animal origin, plant oils of triglyceride type, synthetic triglycerides, fluoro oils, fatty alcohols, fatty acid and/or fatty alcohol esters other than triglycerides and plant waxes, non-silicone waxes and silicones, and mixtures thereof.

It should be remembered that the fatty alcohols, esters and acids more particularly exhibit at least one saturated or unsaturated and linear or branched hydrocarbon-based group which comprises 6 to 30 and better still from 8 to 30 carbon atoms and which is optionally substituted, in particular with one or more hydroxyl groups (in particular 1 to 4). If they are unsaturated, these compounds may comprise one to three conjugated or unconjugated carbon-carbon double bonds.

As regards the C₆-C₁₆ hydrocarbons, they are linear, branched or optionally cyclic, and are preferably alkanes. Mention may be made, by way of example, of hexane, dodecane or isoparaffins, such as isohexadecane or isodecane.

Mention may be made, as hydrocarbon-based oils of animal origin, of perhydrosqualene.

The triglyceride oils of plant or synthetic origin are preferably chosen from liquid fatty acid triglycerides comprising from 6 to 30 carbon atoms, for instance heptanoic or octanoic acid triglycerides, or alternatively, for example, sunflower oil, corn oil, soybean oil, marrow oil, grapeseed oil, sesame seed oil, hazelnut oil, apricot oil, macadamia oil, arara oil, castor oil, avocado oil, caprylic/capric acid triglycerides, for instance those sold by the company Stéarineries Dubois or those sold under the names Miglyol® 810, 812 and 818 by the company Dynamit Nobel, jojoba oil and shea butter oil.

The linear or branched hydrocarbons of mineral or synthetic origin having more than 16 carbon atoms are preferably chosen from liquid paraffins, petroleum jelly, liquid petroleum jelly, polydecenes or hydrogenated polyisobutene, such as Parleam®.

As regards the C₆-C₁₆ alkanes, they are linear, branched or optionally cyclic. By way of example, mention may be made of hexane, dodecane and isoparaffins such as isohexadecane and isodecane.

As oils of animal, plant, mineral or synthetic origin that may be used in the composition of the invention, examples that may be mentioned include:

fluoro oils which may be chosen from perfluoromethylcyclopentane and perfluoro-1,3-dimethylcyclohexane, sold under the names Flutec® PC1 and Flutec® PC3 by the company BNFL Fluorochemicals; perfluoro-1,2-dimethylcyclobutane; perfluoroalkanes such as dodecafluoropentane and tetradecafluorohexane, sold under the names PF 5050® and PF 5060® by the company 3M, or alternatively bromoperfluorooctyl sold under the name Foralkyl® by the company Atochem; nonafluoromethoxybutane and nonafluoroethoxyisobutane; perfluoromorpholine derivatives such as 4-trifluoromethyl perfluoromorpholine sold under the name PF 5052® by the company 3M.

The fatty alcohols which are suitable for the implementation of the invention are more particularly chosen from saturated or unsaturated and linear or branched alcohols comprising from 6 to 30 carbon atoms and preferably from 8 to 30 carbon atoms. Mention may be made, for example, of cetyl alcohol, stearyl alcohol and their mixture (cetearyl alcohol), octyldodecanol, 2-butyloctanol, 2-hexyldecanol, 2-undecylpentadecanol, oleyl alcohol or linoleyl alcohol.

As regards the fatty acid and/or fatty alcohol esters, which are advantageously different from the triglycerides mentioned above, mention may be made in particular of esters of saturated or unsaturated and linear or branched C₁-C₂₆ aliphatic monoacids or polyacids and of saturated or unsaturated and linear or branched C₁-C₂₆ aliphatic monoalcohols or polyalcohols, the total carbon number of the esters being greater than or equal to 6 and more advantageously greater than or equal to 10.

Among the monoesters, mention may be made of dihydroabietyl behenate; octyldodecyl behenate; isocetyl behenate; cetyl lactate; C₁₂-C₁₅ alkyl lactate; isostearyl lactate; lauryl lactate; linoleyl lactate; oleyl lactate; (iso)stearyl octanoate; isocetyl octanoate; octyl octanoate; cetyl octanoate; decyl oleate; isocetyl isostearate; isocetyl laurate; isocetyl stearate; isodecyl octanoate; isodecyl oleate; isononyl isononanoate; isostearyl palmitate; methylacetyl ricinoleate; myristyl stearate; octyl isononanoate; 2-ethylhexyl isononate; octyl palmitate; octyl pelargonate; octyl stearate; octyldodecyl erucate; oleyl erucate; ethyl and isopropyl palmitates, 2-ethylhexyl palmitate, 2-octyldecyl palmitate, alkyl myristates such as isopropyl, butyl, cetyl, 2-octyldodecyl, myristyl or stearyl myristate, hexyl stearate, butyl stearate, isobutyl stearate; dioctyl malate, hexyl laurate, 2-hexyldecyl laurate.

Still within the context of this variant, esters of C₄-C₂₂ dicarboxylic or tricarboxylic acids and of C₁-C₂₂ alcohols and esters of mono-, di- or tricarboxylic acids and of C₂-C₂₆ di-, tri-, tetra- or pentahydroxy alcohols may also be used.

Mention may in particular be made of: diethyl sebacate; diisopropyl sebacate; diisopropyl adipate; di(n-propyl) adipate; dioctyl adipate; diisostearyl adipate; dioctyl maleate; glyceryl undecylenate; octyldodecyl stearoyl stearate; pentaerythrityl monoricinoleate; pentaerythrityl tetraisononanoate; pentaerythrityl tetrapelargonate; pentaerythrityl tetraisostearate; pentaerythrityl tetraoctanoate; propylene glycol dicaprylate; propylene glycol dicaprate; tridecyl erucate; triisopropyl citrate; triisostearyl citrate; glyceryl trilactate; glyceryl trioctanoate; trioctyldodecyl citrate; trioleyl citrate; propylene glycol dioctanoate; neopentyl glycol diheptanoate; diethylene glycol diisononanoate; and polyethylene glycol distearates.

Among the esters mentioned above, use is preferably made of ethyl, isopropyl, myristyl, cetyl or stearyl palmitate, 2-ethylhexyl palmitate, 2-octyldecyl palmitate, alkyl myristates, such as isopropyl, butyl, cetyl or 2-octyldodecyl myristate, hexyl stearate, butyl stearate, isobutyl stearate, dioctyl malate, hexyl laurate, 2-hexyldecyl laurate, isononyl isononanoate or cetyl octanoate.

The composition may also comprise, as fatty ester, sugar esters and diesters of C₆-C₃₀ and preferably C₁₂-C₂₂ fatty acids. It is recalled that the term “sugar” means oxygen-bearing hydrocarbon-based compounds which have several alcohol functions, with or without aldehyde or ketone functions, and which comprise at least 4 carbon atoms. These sugars can be monosaccharides, oligosaccharides or polysaccharides.

Mention may be made, as suitable sugars, for example, of sucrose (or saccharose), glucose, galactose, ribose, fucose, maltose, fructose, mannose, arabinose, xylose and lactose, and derivatives thereof, in particular alkyl derivatives, such as methyl derivatives, for instance methylglucose.

The sugar esters of fatty acids may be chosen in particular from the group comprising the esters or mixtures of esters of sugars described previously and of linear or branched, saturated or unsaturated C₆-C₃₀ and preferably C₁₂-C₂₂ fatty acids. If they are unsaturated, these compounds may comprise one to three conjugated or unconjugated carbon-carbon double bonds.

The esters according to this variant can also be chosen from mono-, di-, tri- and tetraesters, polyesters, and mixtures thereof.

These esters can, for example, be oleates, laurates, palmitates, myristates, behenates, cocoates, stearates, linoleates, linolenates, caprates, arachidonates or mixtures thereof, such as, in particular, oleate/palmitate, oleate/stearate or palmitate/stearate mixed esters.

More particularly, use is made of monoesters and diesters and in particular mono- or di-oleate, -stearate, -behenate, -oleate/palmitate, -linoleate, -linolenate or -oleate/stearate of sucrose, of glucose or of methylglucose.

An example that may be mentioned is the product sold under the name Glucate® DO by the company Amerchol, which is a methylglucose dioleate.

Examples of esters or mixtures of esters of sugar and of fatty acid that may also be mentioned include:
the products sold under the names F160, F140, F110, F90, F70 and SL40 by the company Crodesta, respectively denoting sucrose palmitate/stearates formed from 73% monoester and 27% diester and triester, from 61% monoester and 39% diester, triester and tetraester, from 52% monoester and 48% diester, triester and tetraester, from 45% monoester and 55% diester, triester and tetraester, from 39% monoester and 61% diester, triester and tetraester, and sucrose monolaurate;
the products sold under the name Ryoto Sugar Esters, for example referenced B370 and corresponding to sucrose behenate formed from 20% monoester and 80% diester-triester-polyester;
the sucrose monopalmitate/stearate-dipalmitate/stearate sold by the company Goldschmidt under the name Tegosoft® PSE.

The non-silicone wax(es) are chosen in particular from carnauba wax, candelilla wax, esparto wax, paraffin wax, ozokerite, plant waxes, such as olive tree wax, rice wax, hydrogenated jojoba wax or absolute flower waxes, such as the blackcurrant blossom essential wax sold by the company Bertin (France), or animal waxes, such as beeswaxes or modified beeswaxes (cerabellina); other waxes or waxy starting materials which can be used according to the invention are in particular marine waxes, such as that sold by the company Sophim under the reference M82, polyethylene waxes or polyolefin waxes in general.

The silicones that may be used in the cosmetic compositions of the present invention are volatile or non-volatile, cyclic, linear or branched silicones, which are unmodified or modified with organic groups, having a viscosity from 5×10⁻⁶ to 2.5 m²/s at 25° C., and preferably 1×10⁻⁵ to 1 m²/s.

The silicones which can be used in accordance with the invention can be in the form of oils, waxes, resins or gums.

Preferably, the silicone is chosen from polydialkylsiloxanes, in particular polydimethylsiloxanes (PDMSs), and organomodified polysiloxanes comprising at least one functional group chosen from amino groups and alkoxy groups.

Organopolysiloxanes are defined in greater detail in Walter Noll's “Chemistry and Technology of Silicones” (1968), Academic Press. They can be volatile or non-volatile.

When they are volatile, the silicones are more particularly chosen from those having a boiling point of between 60° C. and 260° C., and more particularly still from:

(i) cyclic polydialkylsiloxanes containing from 3 to 7 and preferably from 4 to 5 silicon atoms. These are, for example, octamethylcyclotetrasiloxane sold in particular under the name Volatile Silicone® 7207 by Union Carbide or Silbione® 70045 V2 by Rhodia, decamethylcyclopentasiloxane sold under the name Volatile Silicone® 7158 by Union Carbide, and Silbione® 70045 V5 by Rhodia, and mixtures thereof. Mention may also be made of cyclocopolymers of the dimethylsiloxane/methylalkylsiloxane type, such as Volatile Silicone® FZ 3109 sold by the company Union Carbide, of formula:

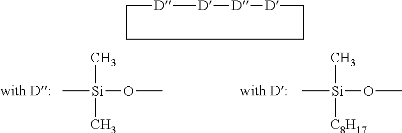

Mention may also be made of mixtures of cyclic polydialkylsiloxanes with organosilicon compounds, such as the mixture of octamethylcyclotetrasiloxane and tetra(trimethylsilyl)pentaerythritol (50/50) and the mixture of octamethylcyclotetrasiloxane and oxy-1,1′-bis(2,2,2′,2′,3,3′-hexatrimethylsilyloxy)neopentane;

(ii) linear volatile polydialkylsiloxanes containing 2 to 9 silicon atoms and having a viscosity of less than or equal to 5×10⁻⁶ m²/s at 25° C. An example is decamethyltetrasiloxane sold in particular under the name SH 200 by the company Toray Silicone. Silicones which fall into this category are also described in the paper published in Cosmetics and Toiletries, Vol. 91, January 76, pp. 27-32, Todd & Byers, “Volatile Silicone Fluids for Cosmetics”.

Use is preferably made of non-volatile polydialkylsiloxanes, polydialkylsiloxane gums and resins, polyorganosiloxanes modified with the organofunctional groups above, and mixtures thereof.

These silicones are more particularly chosen from polydialkylsiloxanes, among which mention may be made mainly of polydimethylsiloxanes having trimethylsilyl end groups. The viscosity of the silicones is measured at 25° C. according to Standard ASTM 445 Appendix C.

Mention may be made, among these polydialkylsiloxanes, without limitation, of the following commercial products:
the Silbione® oils of the 47 and 70 047 series or the Mirasil® oils sold by Rhodia, such as, for example, the oil 70 047 V 500 000;
the oils of the Mirasil® series sold by the company Rhodia;
the oils of the 200 series from the company Dow Corning, such as DC200 with a viscosity of 60 000 mm²/s;
the Viscasil® oils from General Electric and certain oils of the SF series (SF 96, SF 18) from General Electric.

Mention may also be made of polydimethylsiloxanes having dimethylsilanol end groups known under the name dimethiconol (CTFA), such as the oils of series 48 from the company Rhodia.

In this category of polydialkylsiloxanes, mention may also be made of the products sold under the names Abil Wax® 9800 and 9801 by the company Goldschmidt, which are polydi(C₁-C₂₀)alkylsiloxanes.

The silicone gums that may be used in accordance with the invention are in particular polydialkylsiloxanes and preferably polydimethylsiloxanes with high number-average molecular weights of between 200 000 and 1 000 000, used alone or as a mixture in a solvent. This solvent can be chosen from volatile silicones, polydimethylsiloxane (PDMS) oils, polyphenylmethylsiloxane (PPMS) oils, isoparaffins, polyisobutylenes, methylene chloride, pentane, dodecane, tridecane or their mixtures.

Products which can be used more particularly in accordance with the invention are mixtures such as:
the mixtures formed from a polydimethylsiloxane hydroxylated at the chain end, or dimethiconol (CTFA), and from a cyclic polydimethylsiloxane, also known as cyclomethicone (CTFA), such as the product Q2 1401 sold by the company Dow Corning;
the mixtures of a polydimethylsiloxane gum and of a cyclic silicone, such as the product SF 1214 Silicone Fluid from General Electric; this product is an SF 30 gum corresponding to a dimethicone, having a number-average molecular weight of 500 000, dissolved in the oil SF 1202 Silicone Fluid corresponding to decamethylcyclopentasiloxane;
the mixtures of two PDMSs with different viscosities, and more particularly of a PDMS gum and a PDMS oil, such as the product SF 1236 from the company General Electric. The product SF 1236 is a mixture of a gum SE 30 defined above, with a viscosity of 20 m²/s, and of an oil SF 96 with a viscosity of 5×10⁻⁶ m²/s. This product preferably comprises 15% of gum SE 30 and 85% of an oil SF 96.

The organopolysiloxane resins that may be used in accordance with the invention are crosslinked siloxane systems containing the following units:

R₂SiO_{2/2}, R₃SiO_{1/2}, RSiO_{3/2} and SiO_{4/2} in which R represents an alkyl containing 1 to 16 carbon atoms. Among these products, those that are particularly preferred are those in which R denotes a C₁-C₄ lower alkyl group, more particularly methyl.

Mention may be made, among these resins, of the product sold under the name Dow Corning 593 or those sold under the names Silicone Fluid SS 4230 and SS 4267 by the company General Electric, which are silicones of dimethyl/trimethylsiloxane structure.

Mention may also be made of the resins of the trimethyl siloxysilicate type, sold in particular under the names X22-4914, X21-5034 and X21-5037 by the company Shin-Etsu.

The organomodified silicones that can be used in accordance with the invention are silicones as defined above and comprising in their structure one or more organofunctional groups attached via a hydrocarbon-based group.

In addition to the silicones described above, the organomodified silicones can be polydiarylsiloxanes, in particular polydiphenylsiloxanes, and polyalkylarylsiloxanes functionalized by the abovementioned organofunctional groups.

The polyalkylarylsiloxanes are particularly chosen from linear and/or branched polydimethyl/methylphenylsiloxanes and polydimethyl/diphenylsiloxanes with a viscosity ranging from 1×10⁻⁵ to 5×10⁻² m²/s at 25° C.

Mention may be made, among these polyalkylarylsiloxanes, by way of example, of the products sold under the following names:
the Silbione® oils of the 70 641 series from Rhodia;
the oils of the Rhodorsil® 70 633 and 763 series from Rhodia;
the oil Dow Corning 556 Cosmetic Grade Fluid from Dow Corning;
the silicones of the PK series from Bayer, such as the product PK20;
the silicones of the PN and PH series from Bayer, such as the products PN1000 and PH1000;
certain oils of the SF series from General Electric, such as SF 1023, SF 1154, SF 1250 and SF 1265.

Mention may be made, among the organomodified silicones, of polyorganosiloxanes comprising:
substituted or unsubstituted amino groups, such as the products sold under the name GP 4 Silicone Fluid and GP 7100 by the company Genesee or the products sold under the names Q2 8220 and Dow Corning 929 or 939 by the company Dow Corning. The substituted amino groups are, in particular, C₁-C₄ aminoalkyl groups;
alkoxylated groups, such as the product sold under the name Silicone Copolymer F-755 by SWS Silicones, and Abil Wax® 2428, 2434 and 2440 by the company Goldschmidt.

More particularly, the fatty substances are chosen from compounds that are liquid or pasty at ambient temperature (25° C.) and at atmospheric pressure.

Preferably, the fatty substance is a compound that is liquid at a temperature of 25° C. and at atmospheric pressure.

The fatty substances are advantageously chosen from C₆-C₁₆ alkanes, non-silicone oils of plant, mineral or synthetic origin, fatty alcohols, fatty acid and/or fatty alcohol esters, or mixtures thereof.

Preferably, the fatty substance is chosen from liquid petroleum jelly, C₆-C₁₆ alkanes, polydecenes, liquid fatty acid and/or fatty alcohol esters, liquid fatty alcohols, or mixtures thereof.

When they are present, the fatty substances may represent from 0.01% to 95%, preferably from 0.1% to 90% by weight, better still from 0.5% to 80% by weight and even better still from 1% to 30% by weight, relative to the total weight of the composition.

Advantageously, the composition according to the invention may comprise a plasticizer. Such an agent may be chosen from any compound known to those skilled in the art as being capable of fulfilling the desired function. In particular, this agent is chosen from the usual plasticizers or coalescers, such as:
glycols and derivatives thereof such as diethylene glycol ethyl ether, diethylene glycol methyl ether, diethylene glycol butyl ether or alternatively diethylene glycol hexyl ether, ethylene glycol ethyl ether, ethylene glycol butyl ether, ethylene glycol hexyl ether or pentylene glycol,
glycerol esters,
propylene glycol derivatives and in particular propylene glycol phenyl ether, propylene glycol diacetate, propylene glycol butyl ether, tripropylene glycol butyl ether, propylene glycol methyl ether, dipropylene glycol ethyl ether, tripropylene glycol methyl ether, diethylene glycol methyl ether or propylene glycol butyl ether,
acid esters, in particular carboxylic acid esters, such as citrates, phthalates, adipates, carbonates, tartrates, phosphates or sebacates,
mixtures thereof.

The amount of plasticizer can be chosen by those skilled in the art on the basis of their general knowledge, so as to obtain a polymeric system resulting in a film which has the desired mechanical properties. In practice, this amount ranges from 0.01% to 25% of the total weight of the composition and better still from 0.01% to 15%.

The composition of the invention may be aqueous or anhydrous.

In a first variant, if the composition is aqueous, it comprises less than 50% of water, preferably from 5% to 45% of water, better still from 5% to 25% of water and even better still from 5% to 15% by weight of water, relative to the total weight of the composition.

In a second variant, if the composition is aqueous, it comprises at least 50% of water, preferably from 50% to 95% of water, better still from 55% to 90% of water and even better still from 60% to 85% by weight of water, relative to the total weight of the composition.

The term “anhydrous” is intended to mean a composition not containing any added water, i.e. a composition in which the water possibly present comes only from the water of crystallization or of adsorption of the starting materials. In any event, an anhydrous composition contains less than 5% by weight of water and better still less than 1% by weight of water, relative to the total weight of the composition.

Whether it is anhydrous or aqueous, the composition according to the invention may contain one or more organic solvents that are liquid at ambient temperature (25° C.) and at atmospheric pressure (760 mmHg, i.e. 1.013×10⁵ Pa). Besides the silicones and the nonsiliceous liquid fatty substances described above, the liquid organic solvent(s) can be chosen from C₁-C₄ lower alcohols, such as ethanol, isopropanol, tert-butanol or n-butanol, polyols, for instance propylene glycol, polyol ethers, C₃-C₄ ketones, for instance acetone and methyl ethyl ketone, C₁-C₄ alkyl acetates, for instance methyl acetate and ethyl acetate, and mixtures thereof. The solvent is preferably ethanol.

Advantageously, the composition is aqueous or aqueous-alcoholic. When the alcohol is present, its proportion is in particular between 1% and 99% by weight, preferably between 5% and 90% by weight and even more preferentially between 10% and 85% by weight, relative to the total weight of the composition.

The pH of the composition according to the invention, if it is aqueous, generally ranges from 2 to 9 and in particular from 3 to 8. It may be adjusted to the desired value by means of acidifying or basifying agents normally used in the cosmetics industry for this type of application, or alternatively using standard buffer systems.

Among the acidifying agents, examples that may be mentioned include the organic acids already mentioned previously, or inorganic acids.

The term “inorganic acid” is intended to mean any acid derived from an inorganic compound. Among the inorganic acids, mention may be made of hydrochloric acid, orthophosphoric acid, sulfuric acid, sulfonic acids and nitric acid.

Use may be made in particular of inorganic or organic acids such as hydrochloric acid, orthophosphoric acid or sulfuric acid, carboxylic acids, for instance acetic acid, tartaric acid, citric acid or lactic acid, and sulfonic acids.

Among the basifying agents, examples that may be mentioned include aqueous ammonia, alkali metal carbonates, alkanolamines, such as mono-, di- and triethanolamines and derivatives thereof, sodium hydroxide, potassium hydroxide and the compounds of the following formula:

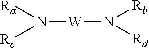

in which W is a propylene residue optionally substituted with a hydroxyl group or a C₁-C₄ alkyl group; Rₐ, R_{b}, R_{c} and R_{d}, which may be identical or different, represent a hydrogen atom or a C₁-C₄ alkyl or C₁-C₄ hydroxyalkyl group.

Preferably, the pH modifiers may be chosen from alkaline agents such as aqueous ammonia, monoethanolamine, diethanolamine, triethanolamine, 1,3-propanediamine or an alkaline hydroxide, such as 2-amino-2-methyl-1-propanol, or else acidifying agents, such as phosphoric acid or hydrochloric acid.

The composition may be in the form of a solution, a dispersion or an emulsion. The polymer may be emulsified as an oil-in-water or water-in-oil emulsion or as a multiple emulsion.

The composition of the invention may be in the form of a foam, a gel, a serum, a cream, a paste, a wax, a liquid lotion or a lacquer.

The composition may be packaged in a pump-dispenser bottle or in an aerosol device.

When it is packaged in an aerosol-type device, the liquid phase/propellant weight ratio of the pressurized composition of the present invention is preferably between 50 and 0.05, and in particular between 50 and 1.

For the aerosol formulations, any liquefiable gas customarily used in aerosol devices will be used as propellant gas.

Use will be made in particular of a propellant chosen from dimethyl ether, C₃-C₅ alkanes, chlorinated and/or fluorinated, halogenated or non-halogenated, volatile hydrocarbons, usually used in aerosol devices. Carbon dioxide, nitrous oxide, nitrogen or compressed air, or mixtures thereof, may also be used as propellant.

Preferably, the compound(s) constituting the propellant used are chosen from non-halogenated C₃-C₅ alkanes, such as propane, n-butane and isobutane, halogenated, and in particular chlorinated and/or fluorinated, C₃-C₅ alkanes, such as 1,1-difluoroethane, and mixtures thereof.

According to a particularly preferred embodiment, the alkane(s) of the propellant gas are nonhalogenated. Even more preferentially, the propellant gas is dimethyl ether or a mixture of propane, n-butane and isobutane.

In the case of aerosol foams, the composition introduced into the aerosol device may, for example, be in the form of a lotion, or dispersions or emulsions which, after dispensing from the aerosol device, form foams to be applied to keratin materials.

These foams must be sufficiently stable so as not to rapidly liquefy and must also rapidly disappear, either spontaneously or during the massaging which is used to cause the composition to penetrate into keratin materials and/or to distribute the composition over keratin materials and more particularly the head of hair and/or the hair.

In the case of aerosol foams, the composition according to the invention may also contain at least one cationic, non-ionic, anionic or amphoteric surfactant as previously described.

The propellant gas is present in the composition according to the invention in proportions preferably ranging from 1% to 99% by weight, more preferentially from 1.5% to 50% by weight and better still from 2% to 30% by weight, relative to the total weight of the composition.

In a first variant, when the propellant is a hydrocarbon or a mixture of hydrocarbons, it is present in the composition in proportions preferably ranging from 0.1% to 60% by weight, more preferentially from 0.5% to 30% by weight and better still from 0.5% to 5% by weight, relative to the total weight of the composition.

In a second variant, when the propellant is a gas which is partially soluble in the composition, such as dimethyl ether or 1,1-difluoroethane, it is present in the composition in proportions preferably ranging from 10% to 50% by weight and more preferentially from 30% to 45% by weight, relative to the total weight of the composition.

The aerosol device used to package the composition of the invention may be made up of two compartments, formed from an outer aerosol can comprising an inner bag hermetically sealed to a valve. The composition is introduced into the inner bag and a compressed gas is introduced between the bag and the can at a pressure sufficient to make the product come out in the form of a spray through a nozzle orifice. Such a device is sold, for example, under the name EP Spray by the company EP-Spray System SA. Said compressed gas is preferably used at a pressure of between 1 and 12 bar and even better still between 9 and 11 bar.

The compositions according to the invention may also contain one or more additional compounds chosen from ceramides and pseudoceramides, vitamins and provitamins, including panthenol, water-soluble or liposoluble, silicone or non-silicone sunscreens, nacreous agents and opacifiers, sequestrants, antifoams, non-fixing cationic polymers, non-ionic fixing polymers, silanes other than those of formula (I), chitosans and derivatives, inorganic and organic thickeners, antioxidants, hydroxy acids, antidandruff agents, anti-seborrhoeic agents, hair-loss counteractants and/or hair restorers, penetrants, fragrances, peptizers, amino acids and preservatives, or any other additive conventionally used in the cosmetics field.

These additives may be present in the composition according to the invention in an amount ranging from 0 to 20% by weight, relative to the total weight of the composition.

Those skilled in the art will take care to select the optional additives and amounts thereof in such a way that they do not interfere with the properties of the compositions of the present invention.

The present invention also relates to a cosmetic hair treatment process, which consists in applying to the hair an effective amount of a composition as described above.

This application may or may not be followed by a rinsing.

When the application of the composition is followed by rinsing, the leave-on time of the composition on the keratin materials ranges from a few seconds to 60 minutes, better still from 5 seconds to 30 minutes and even better still from 10 seconds to 10 minutes.

Whether in rinsed mode or non-rinsed mode, the application of the composition may take place in the presence of heat. The heating device may be a hairdryer, a hood dryer, a curling iron or a flat iron. The heating temperature may be between 40° C. and 220° C., in particular at 200° C.

The application of the composition according to the invention to the hair may take place on dry hair or on wet hair. It may in particular be carried out after a shampooing operation or after a pretreatment at acidic or basic pH.

The application of the composition of the invention may also be followed by a post-treatment at acidic or basic pH, followed in particular by drying at ambient temperature or by drying by supplying heat, using a hairdryer, a hood dryer, a curling iron or a flat iron.

The composition of the invention may be applied to the hair in combination with chemical treatments such as oxidations, reductions, buffer solutions or mechanical treatments.

The examples that follow serve to illustrate the invention without, however, being limiting in nature.

### EXAMPLES

In the following examples, all the amounts are shown as percentage by weight of active material, relative to the total weight of the composition.

The compositions according to the invention are prepared from the compounds indicated in the table below.

Composition 1

| | |
|---|---|
| Octyltriethoxysilane | 1% |
| Vinyl acetate/crotonate/vinyl neodecanoate copolymer⁽¹⁾ | 5% |
| Ethanol | 20% |
| Water | qs 100% |

⁽¹⁾product sold under the name Resyn 28 - 2930 by the company Akzo Nobel.

Composition 2:

| | |
|---|---|
| Octyltriethoxysilane | 10% |
| Vinyl acetate/crotonate/vinyl neodecanoate copolymer⁽¹⁾ | 5% |
| Ethanol | 85% |

⁽¹⁾product sold under the name Resyn 28 - 2930 by the company Akzo Nobel.

Composition 3:

| | |
|---|---|
| Octyltriethoxysilane | 10% |
| n-Octylacrylamide/methyl methacrylate/hydroxypropyl | 5% |
| methacrylate/acrylic acid/t-butyl aminoethyl | |
| methacrylate copolymer ⁽²⁾ | |
| Lactic acid | 2% |
| Water | 5% |
| Ethanol | qs 100% |

⁽²⁾ product sold under the name Amphomer by the company Akzo Nobel.

Composition 4:

| | |
|---|---|
| Octyltriethoxysilane | 10% |
| Acrylate/acrylate hydroxyester copolymer⁽³⁾ | 5% |
| Ethanol | 85% |

⁽³⁾product sold under the name Acudyne 180 Polymer by the company Rohm & Haas.

Composition 5:

| | |
|---|---|
| Octyltriethoxysilane | 1% |
| Amp-acrylates/allyl methacrylate copolymer⁽⁴⁾ | 5% |
| Water | qs 100% |

⁽⁴⁾product sold under the name Fixate G-100L Polymer by the company Lubrizol.

Composition 6

| | |
|---|---|
| Octyltriethoxysilane | 1% |
| Polyvinylpyrrolidone ⁽⁵⁾ | 5% |
| Ethanol | 20% |
| Water | 74% |

⁽⁵⁾ product sold under the name Luviskol ® K30 by the company BASF.

Composition 7:

| | |
|---|---|
| Octyltriethoxysilane | 10% |
| Vinylpyrrolidone/vinyl acetate copolymer (60/40) copolymer⁽⁶⁾ | 5% |
| Ethanol | 85% |

⁽⁶⁾product sold under the name Luviskol ® VA 64 by the company BASF.

Composition 8:

| | |
|---|---|
| Octyltriethoxysilane | 10% |
| Polyvinyl alcohol⁽⁷⁾ | 5% |
| Ethanol | 80% |
| Water | 5% |

⁽⁷⁾product sold under the name Celvol 205 PV Alcohol by the company Sekisui Specialty Chemicals.

Compositions 1 to 8 are applied to dry hair at ambient temperature and without rinsing.

After drying, good form retention of the hairstyle is obtained, with no introduction of a greasy appearance or feel. In addition, the fibres have a soft natural feel and are smooth after disentangling.

1.-17. (canceled)

18. A cosmetic composition comprising:
(i) at least one fatty-chain silane of formula (I) and/or oligomers thereof:
R₁Si(OR₂)_{z}(R₃)ₓ(OH)_{y} (I)
wherein:
R₁ is chosen from linear or branched alkyl or alkenyl groups comprising from 7 to 18 carbon atoms;
R₂ and R₃, which may be identical or different, are chosen from linear or branched alkyl groups comprising from 1 to 6 carbon atoms;
y is equal to an integer ranging from 0 to 3;
z is equal to an integer ranging from 0 to 3; and
x is equal to an integer ranging from 0 to 2,
wherein the sum z+x+y is equal to 3; and
(ii) at least one fixing polymer chosen from anionic, amphoteric, and non-ionic fixing polymers,
wherein said at least one fatty-chain silane of formula (I) and/or oligomer thereof is present in the composition in an amount ranging from about 0.5% to about 15% by weight, relative to the total weight of the composition.

19. The composition according to claim 18, wherein R₂ is an alkyl group comprising from 1 to 4 carbon atoms, and R₃ is an alkyl group comprising from 1 to 4 carbon atoms.

20. The composition according to claim 18, wherein R₁ is an alkyl group.

21. The composition according to claim 18, wherein z is equal to an integer ranging from 1 to 3.

22. The composition according to claim 18, wherein the at least one fatty-chain silane of formula (I) and/or oligomer thereof is chosen from octyltriethoxysilane, dodecyltriethoxysilane, octadecyltriethoxysilane, and hexadecyltriethoxysilane.

23. The composition according to claim 18, wherein the at least one fatty-chain silane of formula (I) and/or oligomer thereof is present in an amount ranging from about 1% to about 15% by weight, relative to the total weight of the composition.

24. The composition according to claim 18, wherein the at least one anionic, amphoteric, and non-ionic fixing polymer is chosen from homopolymers or copolymers of acrylic and methacrylic acid or salts thereof; crotonic acid copolymers; copolymers of C₄-C₈ monounsaturated carboxylic acids or anhydrides; polyacrylamides comprising carboxylate groups; polyurethanes optionally comprising silicones, with carboxylate groups; homopolymers or copolymers comprising sulfonic groups; copolymers comprising acidic vinyl units and basic vinyl units; crosslinked and acylated polyaminoamides; polymers comprising zwitterionic units; polymers derived from chitosan; modified (C₁-C₅)alkyl vinyl ether/maleic anhydride copolymers; and polymers comprising units deriving from
a) at least one monomer chosen from acrylamides or methacrylamides, optionally substituted on the nitrogen atom with an alkyl group,
b) at least one acidic comonomer containing at least one reactive carboxylic groups, and
c) at least one basic comonomer chosen from anionic polymers comprising carboxylic groups and crotonic acid copolymers, polyalkyloxazolines, vinyl acetate homopolymers, vinyl acetate copolymers, ester homopolymers and copolymers, copolymers of acrylonitrile and of a non-ionic monomer, styrene homopolymers, styrene copolymers, polyamides, vinyllactam homopolymers, vinyllactam copolymers, non-hydrolyzed polyvinylformamide polymers, or polyvinyl alcohols.

25. The composition according to claim 18, wherein the anionic, amphoteric, or non-ionic fixing polymer is present in an amount ranging from about 0.1% to about 50% by weight, relative to the total weight of the composition.

26. The composition according to claim 18, wherein the weight ratio between the amount of at least one silane of formula (I) and/or oligomer thereof and the amount of anionic, amphoteric, or non-ionic fixing polymer ranges from about 0.05 to about 20.

27. The composition according to claim 18, further comprising at least one additives chosen from cationic, anionic, non-ionic, amphoteric and/or zwitterionic surfactants; silanes other than those of formula (I); fatty substances; plasticizers; water-soluble solvents; and additional fixing polymers other than the at least one anionic, amphoteric, and non-ionic fixing polymer.

28. The composition according to claim 18, wherein the composition is anhydrous or aqueous.

29. The composition according to claim 18, further comprising at least one propellant gas.

30. The composition according to claim 18, further comprising a basifying agent, an acidifying agent, or a buffer.

31. A process for treating hair comprising applying a composition to the hair, said composition comprising:
(i) at least one fatty-chain silane of formula (I) and/or oligomers thereof:
R₁Si(OR₂)_{z}(R₃)ₓ(OH)_{y} (I)
wherein:
R₁ is chosen from linear or branched alkyl or alkenyl groups comprising from 7 to 18 carbon atoms;
R₂ and R₃, which may be identical or different, are chosen from linear or branched alkyl groups comprising from 1 to 6 carbon atoms;
y is equal to an integer ranging from 0 to 3;
z is equal to an integer ranging from 0 to 3; and
x is equal to an integer ranging from 0 to 2,
wherein the sum z+x+y is equal to 3; and
(ii) at least one fixing polymer chosen from anionic, amphoteric, and non-ionic fixing polymers,
wherein said at least one fatty-chain silane of formula (I) and/or oligomer thereof is present in the composition in an amount ranging from about 0.5% to about 15% by weight, relative to the total weight of the composition.

32. A process according to claim 31, wherein applying said composition to the hair occurs in the presence or absence of heat.

33. A cosmetic composition comprising:
(i) octyltriethoxysilane, and
(ii) at least one fixing polymer chosen from homopolymers or copolymers of acrylic and methacrylic acid or salts thereof; crotonic acid copolymers; copolymers of C₄-C₈ monounsaturated carboxylic acids or anhydrides; polyacrylamides comprising carboxylate groups; polyurethanes optionally comprising silicones, with carboxylate groups; homopolymers or copolymers comprising sulfonic groups; copolymers comprising acidic vinyl units and basic vinyl units; crosslinked and acylated polyaminoamides; polymers comprising zwitterionic units; polymers derived from chitosan; modified (C₁-C₅)alkyl vinyl ether/maleic anhydride copolymers; and polymers comprising units deriving from
a) at least one monomer chosen from acrylamides or methacrylamides, optionally substituted on the nitrogen atom with an alkyl group,
b) at least one acidic comonomer containing at least one reactive carboxylic group, and
c) at least one basic comonomer chosen from anionic polymers comprising carboxylic groups and crotonic acid copolymers, polyalkyloxazolines, vinyl acetate homopolymers, vinyl acetate copolymers, ester homopolymers and copolymers, copolymers of acrylonitrile and of a non-ionic monomer, styrene homopolymers, styrene copolymers, polyamides, vinyllactam homopolymers, vinyllactam copolymers, non-hydrolyzed polyvinylformamide polymers and polyvinyl alcohols;
wherein the octyltriethoxysilane is present in an amount ranging from about 0.5% to about 15% by weight, relative to the total weight of the composition, and
further wherein the at least one fixing polymer is present in an amount ranging from about 0.1% to about 50% by weight, relative to the total weight of the composition.
